(19) United States
(12) Patent Application Publication
(10) Pub. No.: US 2025/0155373 A1
(43) Pub. Date: May 15, 2025

(54) HIGHLY FLUORESCENT CONJUGATED PLYMER AND EXPOLOSIVE VAPOR SENSOR USING THE SAME

(71) Applicant: EXSOL Co., Ltd., Gwangju (KR)

(72) Inventors: Honglae SOHN, Gwangju (KR); Kyungkuk KOH, Mokpo-si, Jeollanam-do (KR)

(21) Appl. No.: 18/746,199

(22) Filed: Jun. 18, 2024

(30) Foreign Application Priority Data
Nov. 13, 2023 (KR) 10-2023-0156790

(51) Int. Cl. G01N 21/64; G01N 33/00

(52) U.S. Cl. CPC G01N 21/643; G01N 33/0057; G01N 2021/6439

(57) ABSTRACT

The present invention relates to a highly fluorescent conjugated silole polymer and an explosive vapor sensor using the same. The highly fluorescent conjugated silole polymer is a rigid 2,5-polysilole-based conjugated polymer having benzene subunits containing sterically hindered functional groups (X) at the 2,5 positions of the silole backbone. The penetration of explosive vapors is facilitated by the cavity structure of the silole polymer due to aggregation-induced emission (AIE) and intramolecular charge transfer (ICT) effects. The fluorescent conjugated silole polymer exhibits a fluorescence quenching process upon exposure to explosive vapors and a photoluminescence (PL) recovery process upon removal of explosive vapors, indicating that it may be applied as an explosive vapor sensor capable of real-time detection.

### STATEMENT DESIGNATING GRACE PERIOD INVENTOR DISCLOSURES

Applicant informs that the subject matter of this patent application was disclosed by the inventor or joint inventor or by another who obtained the subject matter disclosed directly or indirectly from the inventor or joint inventor one year or less than before the effective filing date of a claimed invention which do not qualify as prior art under 35 U.S.C. 102(b)(1) for the following non-patent literature.
Detection of TNT Vapors by Fluorescence Quenching and Self-Recovery Using Highly Fluorescent Conjugated Silole Polymers, Honglae Sohn, et al. Macromolecules 2023, 56, 6396-6406 (Aug. 4, 2023).

### BACKGROUND OF THE INVENTION

### Field of the Invention

The present invention relates to a highly fluorescent conjugated silole polymer and an explosive vapor sensor using the same, and more particularly, to the highly fluorescent conjugated silole polymer having benzene subunits containing sterically hindered functional groups (X) at the 2,5 positions of the silole backbone, which promote explosive vapor permeability through the cavity structure of the silole polymer in the solid state, and in particular, is capable of detection of nitroaromatic explosive vapors, and exhibits a fluorescence-quenching process upon exposure to explosive vapors and a photoluminescence (PL) recovery process upon removal of explosive vapors, which are repeated in real time, and an explosive vapor sensor using the same.

### Description of the Related Arts

The detection of ultratrace nitroaromatic explosives (NAEs) such as 2,4,6-trinitrotoluene (TNT) and picric acid (PA) has become increasingly critical due to rising concerns over international terrorism and environmental impact. To enhance detection sensitivity, new materials and transduction methods are required.

Conjugated polymers have emerged as candidate materials for NAE detection, offering the potential for signal amplification and well-defined morphology structures. The electron transport properties of conjugated polymers are particularly relevant for fluorescence-based sensors, as they can enhance sensitivity signals through electron-transfer fluorescence.

As representative conjugated polymers, siloles have garnered attention as promising π-electronic materials, which may find applications in photovoltaic cells, light-emitting diodes (LEDs), flexible memory storage, and chemical sensors, owing to their exceptional photoluminescence and electroluminescence properties.

Siloles, characterized by their five-membered rings with σ*-π* conjugation between the silicon atom and butadiene moiety, possess increased electron affinities and lowered LUMO energy levels due to conjugation. This unique electronic structure enables siloles to exhibit AIE in aggregate states by restricting intramolecular rotations of the phenyl moiety.

Since the aggregation-induced emission enhancement (AIEE) properties of siloles were reported in Non-Patent Document 1, interest in conjugated polymers with AIE properties has increased significantly, and extensive research has focused on developing new AIE chromophores that exhibit significantly higher emission in aggregated states compared to dilute solutions.

Patent Document 1 relates to an organic optical semiconductor chemical sensor for heavy metal detection containing nanoaggregates or nanoparticles of polymetallole, a silole-germole copolymer or a metallole-silane copolymer. According to Patent Document 1, it is possible to detect chromium (VI) and arsenic (V), which are ultra-trace amounts of carcinogenic heavy metals present in drinking water, immediately on site by measuring the degree of quenching of the photoluminescence of the nanoaggregates or nanoparticles whose photoluminescence has been amplified compared to that of the molecular states of polymers, and easy-to-operate and expensive materials are used.

Patent Document 2 utilizes the optical properties of distributed Bragg reflection-type porous silicon whose reflection peak in a desired wavelength range has been adjusted by chemically etching the surface of porous silicon (PSi), wherein polysilole, a photoluminescent polymer solution, is applied to the lower surface of the porous silicon film.

The siloles used in Patent Document 1 or 2 are 1,1-polysilols or a polysilole solution. While the 1,1-polysiloles exhibit aggregation-induced emission enhancement (AIEE) in a water environment, they possess a helical structure as confirmed in a space-filling model of 1,1-polysilole polymers shown in FIG. 1. The helical structure of 1,1-polysiloles lacks a porous morphology that allows analyte intercalation. Thus, the helical structure of 1,1-polysiloles limits their potential for sensing applications in the solid state.

Accordingly, the inventors of the present invention have made efforts to solve the above-described problems occurring in the related art, and as a result, have designed a novel conjugated silole polymer having benzene subunits containing sterically hindered functional groups (X) at the 2,5 positions of the silole backbone, and have found that a rigid 2,5-polysilole-based conjugated polymer having cavities in the solid-state silole polymer structure have high fluorescence properties not only in the solution state but also in the solid state, and in particular, facilitates the penetration of the analyte explosive vapor in the solid state compared to the solution state, and exhibits excellent sensing performance upon removal of the silole polymer to explosive vapors, and fast fluorescence recovery upon removal of explosive vapors, thereby completing the present invention.

### PRIOR ART DOCUMENTS

### [Patent Documents]

(Patent Document 0001) Korean Patent No. 0700161 (published on Mar. 27, 2007, entitled “Organic optical semiconductor chemical sensor for heavy metal detection containing nanoaggregates or nanoparticles of polymetalol, silole-germole copolymer or metallole-silane copolymer”)
(Patent Document 0002) Korean Patent Application Publication No. 2022-0065304 (published on May 20, 2022, entitled “Method for manufacturing porous silicone film and distributed Bragg reflector porous silicon film-based chemical sensor for explosive detection)

### [Non-Patent Documents]

(Non-Patent Document 0001) Aggregation-Induced Emission of 1-Methyl-1,2,3,4,5-Pentaphenylsilole. Chem. Commun. 2001, 18, 1740-1741.

### SUMMARY OF THE INVENTION

An object of the present invention is to provide a novel highly fluorescent conjugated silole polymer.

Another object of the present invention is to provide a nitroaromatic explosive vapor sensor using the highly fluorescent conjugated silole polymer.

To achieve the above objects, the present invention provides a highly fluorescent conjugated silole polymer based on a rigid 2,5-polysilole having a cavity structure in the silole polymer backbone.

Preferably, the present invention provides a highly fluorescent conjugated silole polymer represented by Formula 1 below, which has benzene subunits containing sterically hindered functional groups (X) at the 2,5 positions of the silole backbone.

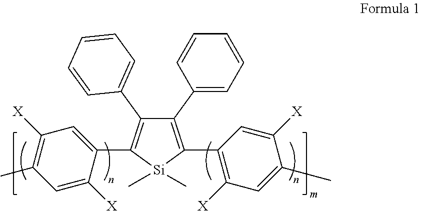

wherein X is a C₂-C₁₂ alkyl group or a C₂-C₁₂ alkoxy group, m is 2 to 20, and n is 1 to 5.

In an embodiment of the present invention, the benzene subunits containing sterically hindered functional groups (X) are octyloxybenzene subunits.

The highly fluorescent conjugated silole polymer is a 2,5-polysilole polymer having a semicrystalline structure, which has a cavity structure in the solid state and thermal stability.

The present invention also provides an explosive vapor sensor using the highly fluorescent conjugated silole polymer.

It provides an explosive vapor detection sensor in which the fluorescence quenching process upon exposure to explosive vapors and the photoluminescence (PL) recovery process upon removal of explosive vapors are repeated in real-time, and it is particularly useful for detecting nitroaromatic explosives (NAEs).

As the present invention provides a rigid 2,5-polysilole-based conjugated polymer having benzene subunits containing sterically hindered functional groups (X) at the 2,5 positions of the silole backbone, compared to 1,1-polysilole known as an explosive sensor, the penetration of the analyte explosive vapor is facilitated by cavities formed in the solid-state silole polymer structure, and in particular, the polysilole-based conjugated polymer is useful for detecting vapors of nitroaromatic explosives (NAEs).

The rigid 2,5-polysilole-based conjugated polymer of the present invention has a cavity structure in the solid-state silole polymer and binding strength with explosive vapors. Therefore, it can provide an explosive vapor detection sensor capable of real-time detection, where fluorescence quenching upon exposure to explosive vapors and rapid photoluminescence (PL) recovery processes upon removal of explosives are reproduced.

### BRIEF DESCRIPTION OF THE DRAWINGS

FIG. 1 shows a space-filling model of a conventional 1,1-polysilole polymer.

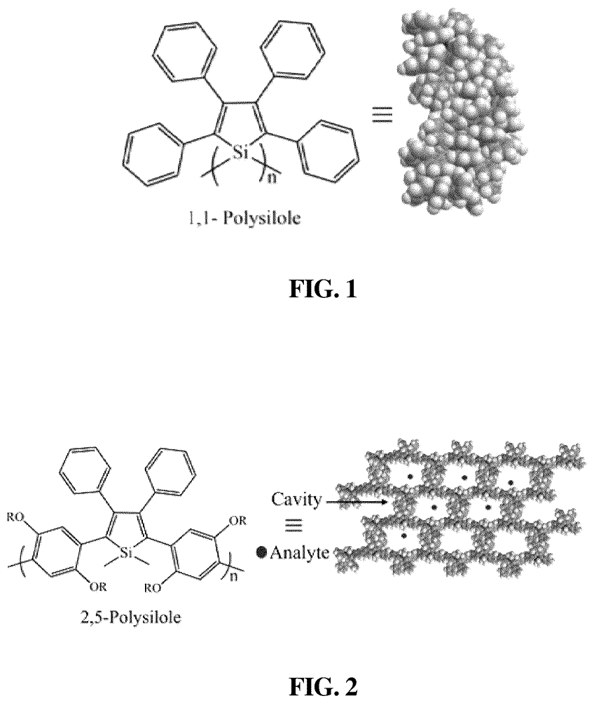

FIG. 2 shows a space-filling model of the 2,5-polysilole polymer of the present invention.

FIG. 3 shows the UV-Vis spectra of a 2,5-polysilole P1 polymer in THF/water solvent conditions with water fractions of 0%, 60%, and 90%.

FIG. 4 shows the UV-Vis spectra of a 2,5-polysilole P2 polymer in THF/water solvent conditions with water fractions of 0%, 60%, and 90%.

FIG. 5 shows the PL spectra of the 2,5-polysilole P1 polymer of the present invention in THF/H₂O mixtures with different water fractions.

FIG. 6 shows the change in relative PL intensity (I/I₀) depending on the water fractions shown in FIG. 5.

FIGS. 7(A) and 7(B) show dynamic light scattering (DLS) measurements for the 2,5-polysilole P1 and P2 polymers of the present invention, and FIGS. 7(C) and 7(D) are scanning electron microscope (SEM) morphologies of the 2,5-polysilole P1 and P2 polymers.

FIG. 8 shows fluorescence images of the 2,5-polysilole P1 polymer of the present invention under 365 nm UV and sunlight conditions in various solvent conditions.

FIG. 9 shows the powder X-ray diffraction (PXRD) spectra of the 2,5-polysilole P1 and P2 polymer powders of the present invention.

FIG. 10 shows the thermal properties of the 2,5-polysilole P1 and P2 polymer powders of the present invention using thermogravimetric analysis (TGA).

FIG. 11 shows the real-time fluorescence intensity profiles of the 2,5-polysilole P1 polymer of the present invention for TNT vapors.

FIGS. 12(A)-12(B) show the PL spectra of the 2,5-polysilole P1 polymer nanoaggregates of the present invention according to the concentration of explosives (a) TNT or (b) PA.

FIGS. 13(A)-13(B) show the PL spectra of the 2,5-polysilole P1 polymer of the present invention in the THF solution state depending on the explosive TNT concentration (a) or PA concentration (b).

FIGS. 14(A)-14(B) show the fluorescence lifetime of the 2,5-polysilole P1 polymer of the present invention in the (a) THF solution and (b) THF/H₂O (f_{w}—99 vol %) nanoaggregate state.

### DETAILED DESCRIPTION OF THE INVENTION

Hereinafter, the present invention will be described in detail.

The present invention provides a highly fluorescent conjugated silole polymer based on a rigid 2,5-polysilole having a cavity structure in the silole polymer backbone.

More specifically, the present invention provides a highly fluorescent conjugated silole polymer represented by Formula 1 below, which has benzene subunits containing sterically hindered functional groups (X) at the 2,5 positions of the silole backbone:

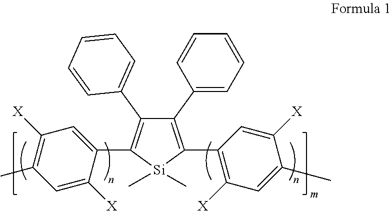

wherein X is a C₂-C₁₂ alkyl group or a C₂-C₁₂ alkoxy group, m is 2 to 20, and n is 1 to 5.

In a preferred example of the present invention, 2,5-polysilole P1 (X=C₈ alkoxy, n=2, and m=10) and 2,5-polysilole P2 (X=C₈ alkoxy, n=3, and m=10), in which X has an octyloxy group, were synthesized, and the fluorescence characteristics and AIEE and ICT properties thereof were investigated.

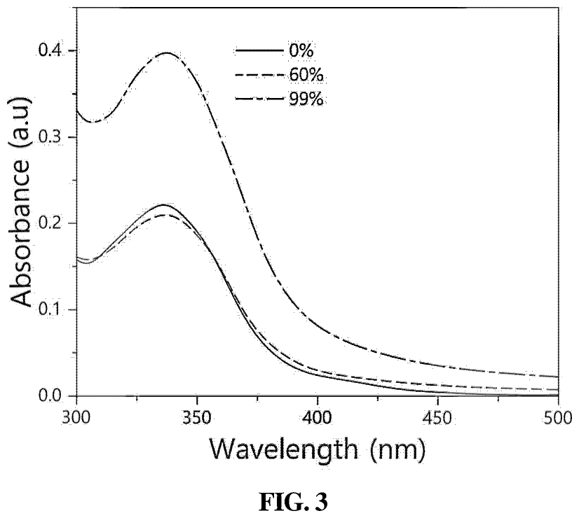

FIG. 2 shows the space-filling model of the 2,5-polysilole polymer of the present invention. Due to the sterically hindered functional groups (X) containing benzene subunits at the 2,5 positions of the silole, cavities are formed in the structure of the solid-state silole polymer, and this porous structure facilitates the penetration of analyte explosive vapors.

In addition, the 2,5-polysilol polymer of the present invention exhibits high solubility in organic solvents such as tetrahydrofuran (THF), toluene, CH₂Cl₂, and CHCl₃, owing to the presence of a large number of free spaces within the polymer repeat units along with the octyloxy chains.

Spectroscopic results of the UV-VIS absorption and fluorescence properties of the 2,5-polysilole P1 or P2 polymers and 1,1-polysilole of the embodiment in various states such as solution, film, and aggregates [Table 1] show the differences in optical properties and emission behavior between polymers in different states. The 2,5-polysilole P1 or P2 polymers have consistent maximum absorption wavelengths (λ_{ab}) due to the same polymer backbone structure, but the maximum emission wavelength (λₑₘ) of the fluorescence spectrum shows a red-shift behavior according to the solution-film-aggregate state. In particular, the emission peak of the P1 polymer showed a red-shift of 93 nm in the solid state compared to the THF solution and exhibited a high absolute quantum yield (QY) value of 44% in the solid state, surpassing the result of 1,1-polysilole (31.9%).

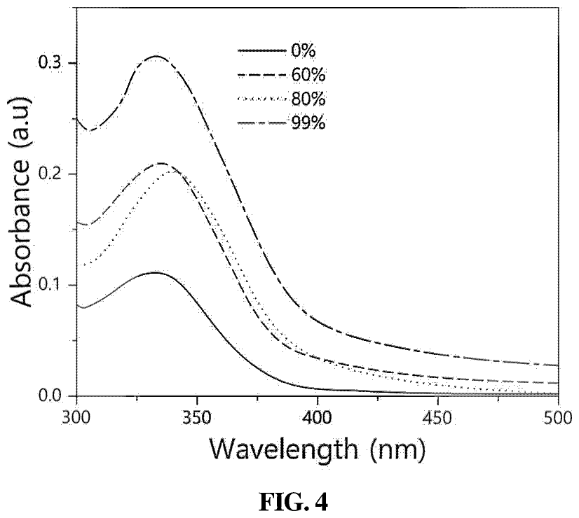

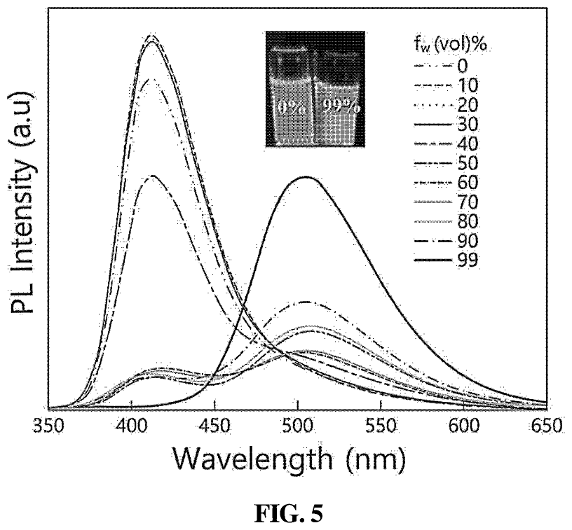

FIGS. 3 and 4 show the UV-Vis spectra of the 2,5-polysilole P1 and P2 polymers of the present invention in THF/H₂O solvents conditions with different water fractions. As shown therein, in the UV-Vis absorption spectra, both the P1 and P2 polymers did not exhibit significant changes in the maximum absorption wavelength, but as the water fraction increased, the absorption intensity increased, confirming that polymer aggregation was formed in the presence of water.

This aggregation process leads to the formation of larger structures or assemblies of polymer molecules, enhancing the absorption intensity. This phenomenon is consistent with the concept of AIEE, where the fluorescence properties of specific molecules are enhanced upon aggregation.

The water-induced aggregation of P1 and P2 at high water fractions is due to the hydrophobic interactions between polymer chains and the exclusion of water molecules in the hydrophobic regions, promoting the formation of larger aggregate structures by the polymer chains and increasing the absorption intensity.

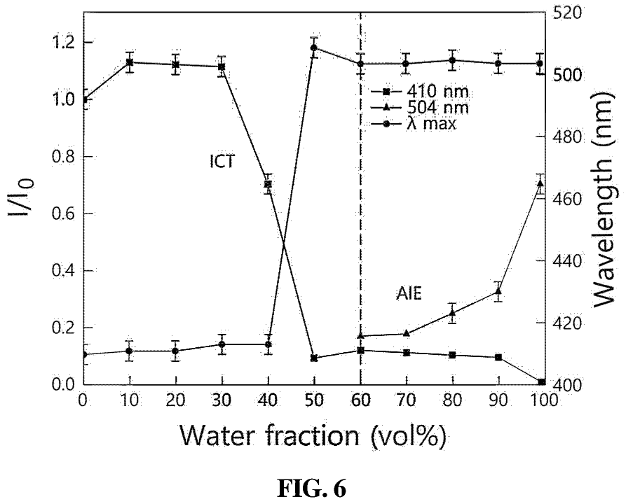

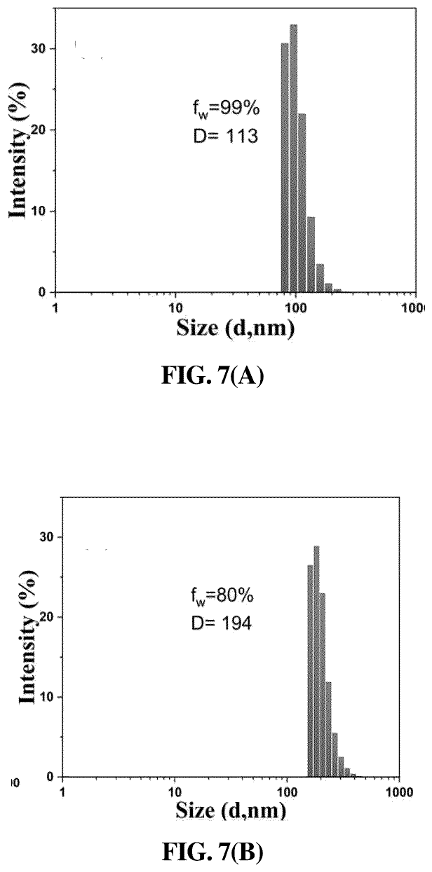

FIGS. 5 and 6 show the PL spectra of the 2,5-polysilole P1 polymer of the present invention in THF/H₂O mixtures with different water fractions, and the change in relative PL intensity (I/I₀) depending on the water fractions. The insets in the figures show fluorescence images of the polymer taken under a 365 nm UV lamp according to THF/H₂O fraction.

The results in FIGS. 5 and 6 show that as the water fraction (f_{w}) increases, the P1 polymer aggregates and exhibits emission peak changes. As the solvent polarity increases, the ICT effect becomes more prominent in P1. This result was evident from the solvatochromic properties observed, where the emission color of the polymer changed when adding water to the solution. These results show that P1 exhibits dual active properties of AIEE and ICT depending on the solvent polarity and aggregation state, and both phenomena contribute to the emission behavior of the polymer.

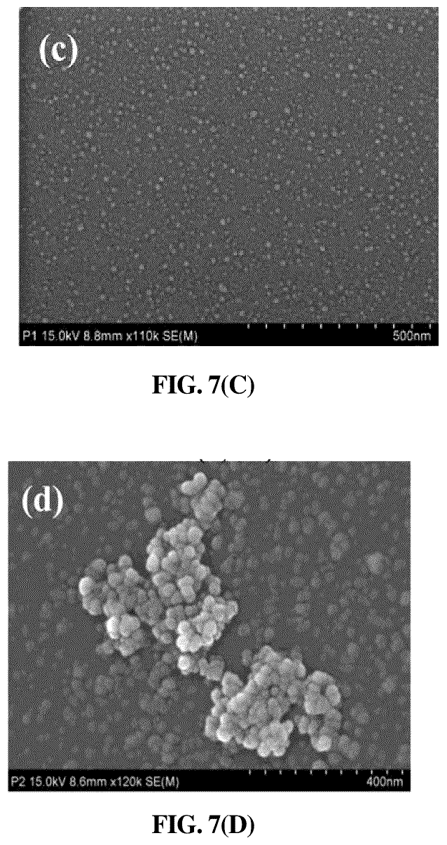

FIGS. 7(A)-7(D) shows dynamic light scattering (DLS) measurements and scanning electron microscope (SEM) morphologies of the 2,5-polysilole P1 and P2 polymers of the present invention. FIGS. 7(A) and 7(B) show dynamic light scattering (DLS) measurements for (A) 2,5-polysilole P1 polymer and (B) 2,5-polysilole P2 polymer, and FIGS. 7(C) and 7(D) show scanning electron microscope (SEM) morphologies of the 2,5-polysilole P1 polymer (C) and the 2,5-polysilole P2 polymer.

According to the DLS result, the average diameters of the particles were about 113 nm for P1 aggregates in f_{w}=99% and 194 nm for P2 aggregates in f_{w}=80%, respectively, indicating that, as the water fraction increased, the particle size of polymer aggregates decreased. In addition, SEM images of (c) P1 and (d) P2 aggregates in THF/H2O mixtures confirm the formation of well-dispersed spherical nanoparticles in aqueous solution for both polymers.

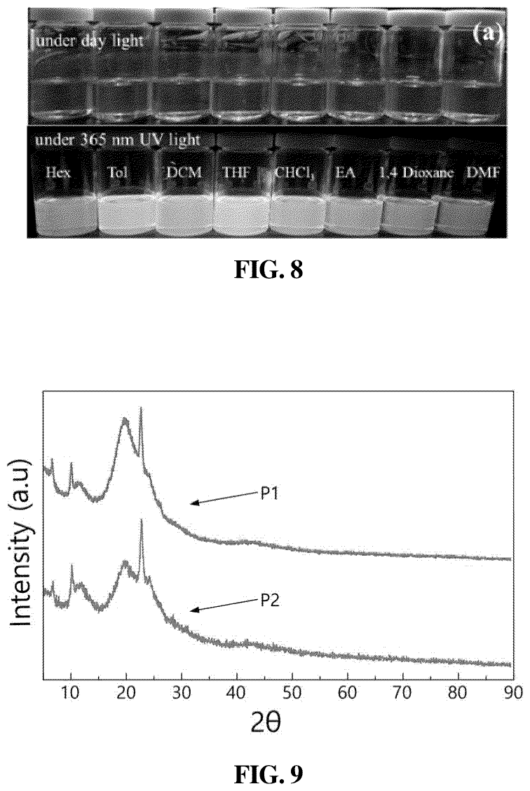

FIG. 8 shows fluorescence images of the 2,5-polysilole P1 polymer of the present invention under 365 nm UV and sunlight in various solvent conditions, providing a visual representation of its emission behavior in different solvent environments.

In addition, fluorescence images of the 2,5-polysilole P2 polymer of the present invention under 365 nm UV and sunlight in various solvent conditions provide a visual representation of its emission behavior in different solvent environments [not shown].

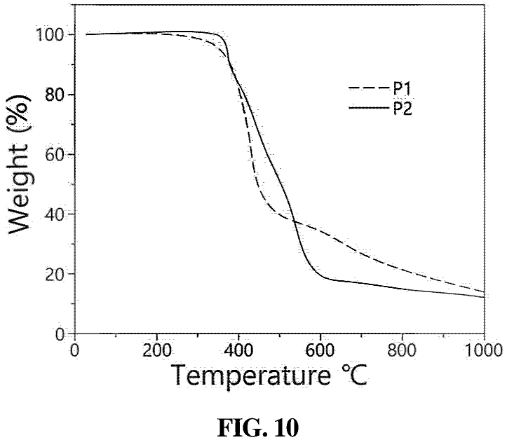

FIG. 9 shows the powder X-ray diffraction (PXRD) spectra of the P1 and P2 polymer powders of the present invention. As shown therein, both exhibiting strong and sharp peaks. These results indicate that both polymers have semi-crystalline structures. Specifically, the regularly repeating units in the polymer structure contribute to the formation of crystalline regions, and the broad peaks indicate the presence of amorphous components. The broad peak width indicates the existence of regions with disordered or less defined molecular arrangements within the polymer.

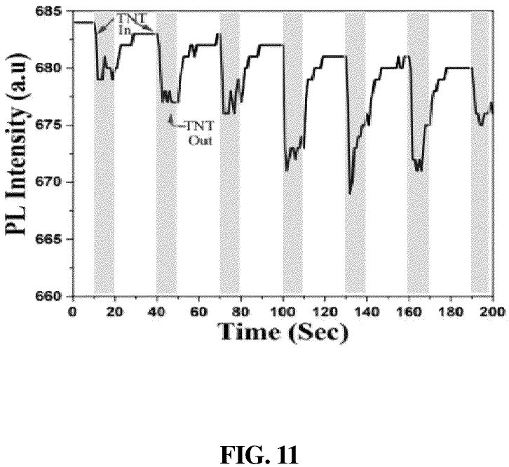

FIG. 10 shows the thermal properties of the P1 and P2 polymer powders of the present invention. As shown therein, both P1 and P2 polymers showed high thermal stability, as almost no weight loss was observed up to a temperature of approximately 300° C.

Thus, the 2,5-polysilole polymers of the present invention can withstand high temperatures and may be suitable for use in various luminescent devices, and the thermal behavior of the polymers is important for ensuring the stability and longevity of devices including the polymers.

Furthermore, the present invention provides an explosive vapor sensor using the highly fluorescent conjugated silole polymer.

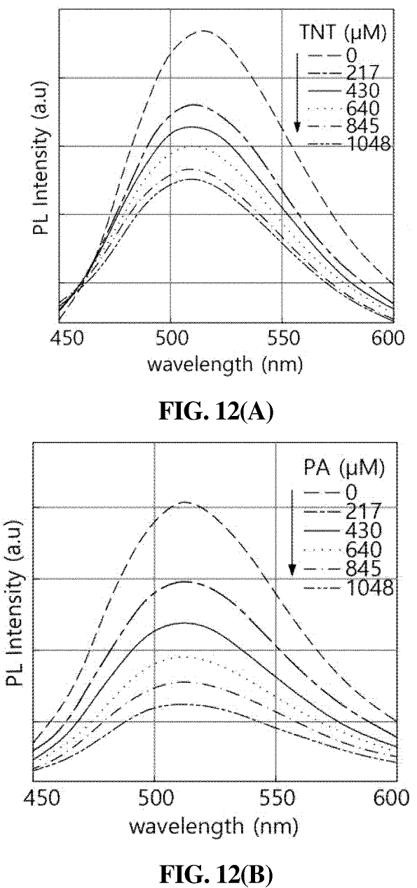

Upon contact with explosive vapors, sensing of the explosive vapors in the solid state can be confirmed by fluorescence quenching of the highly fluorescent conjugated silole polymer. FIG. 11 shows the real-time fluorescence intensity profiles of the 2,5-polysilole P1 polymer of the present invention polymer P1 in response to TNT vapor, depicting the quenching (fluorescence intensity decrease) and recovery (fluorescence intensity increase) processes repeated 7 times over 200 seconds. The arrows indicate the points when the TNT vapor causing quenching is introduced and removed. Specifically, when exposed to 5.5 ppb of TNT vapor for 10 seconds at 25° C., the fluorescence intensity decreases in the quenching stage.

In general, when the binding forces between the polymer and analyte are strong, they prevent the escape of the analyte molecules from the polymer matrix, and thus can lead to irreversible quenching or very slow fluorescence recovery.

While 1,1-polysilole show minimal decreases in fluorescence intensity and require a considerably longer period to recover, the P1 polymer film of the present invention has a very weak binding affinity for TNT vapors and exhibits a semi-reversible response to TNT vapors or a very fast self-recovery process.

Thus, in the case of the 2,5-polysilols polymer of the present invention and explosive (TNT) vapor, the porous space due to the pore structure of the polymer and the TNT vapor interact with relatively weak binding forces, indicating that the 2,5-polysilole polymer can successfully detect TNT vapors on the film and can exhibit efficient fluorescence quenching and rapid recovery of PL intensity upon TNT removal.

From the above-described results, the highly fluorescent conjugated silole polymer of the present invention exhibits a fluorescence quenching process upon exposure to explosive (TNT) vapors and a fast photoluminescence (PL) recovery process upon removal of explosive (TNT) vapors, suggesting that it can be applied as an explosive vapor sensor capable of real-time detection.

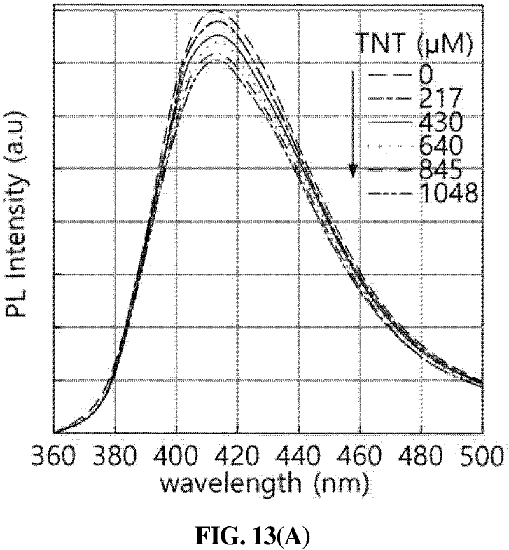

In addition, the quenching behavior of the polymer was investigated for picric acid (PA) and TNT in both THF solution and aggregated states to investigate sensing performance for explosive vapors. FIGS. 12(A)-12(B) show the PL spectra of the 2,5-polysilole P1 polymer of the present invention in the nanoaggregate state depending on the explosive (a) TNT concentration or (b) PA concentration. As can be seen therein, as the concentration of TNT or PA increased, the PL spectrum intensity decreased, indicating that the polymer exhibited PL quenching behavior.

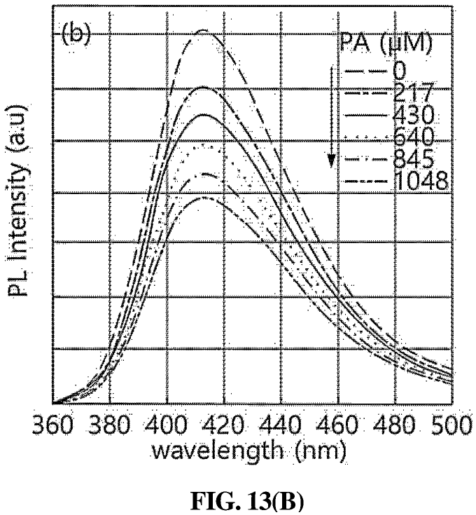

FIGS. 13(A)-13(B) shows the PL spectra of the 2,5-polysilole P1 polymer of the present invention depending in the solution state on the explosive (a) TNT concentration or (b) PA concentration. As can be seen therein, the polymer exhibited quenching behavior depending on the concentration of TNT or PA, and the PL spectrum intensity tended to decrease as the concentration increased.

In the embodiment of the present invention, nitroaromatic explosives (NAE), including 2,4,6-trinitrotoluene (TNT) and picric acid (PA), are described as examples of explosives, but the present invention is not limited thereto.

Specifically, in addition to examples of the nitroaromatic explosives of the present invention, DNT (dinitrotoluene), TATB (triaminotrinitrobenzene or 2,4,6-triamino-1,3,5-trinitrobenzene), HNS (hexanitrostilbene), and tetryl may be included. In addition to the above-mentioned group of nitroaromatic explosives, at least one selected from the group consisting of cyclic nitramine-based compounds, non-cyclic nitramine-based compounds, and aromatic nitrogen heterocyclic compounds may be included.

FIGS. 12(A)-12(B) and 13(A)-13(B) are shown using the Stern-Volmer constant. The lower Stern-Volmer constant values of the 2,5-polysilole P1 and P2 polymers than 1,1-polysiloles may explain the non-flexibility of the P1 and P2 polymers, and the binding affinity between the polysilole and the explosive (TNT or PA) is very weak, which leads to reversible quenching or very fast recovery.

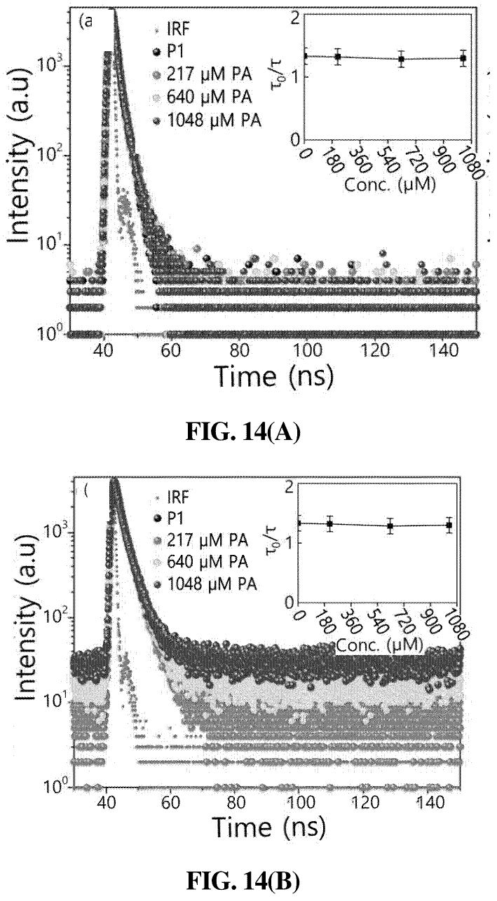

FIGS. 14(A)-14(B) show the results of observing the fluorescence lifetime of the 2,5-polysilole P1 polymer of the present invention in the THF solution state (a) and the THF/H₂O (f_{w}-99 vol %) nanoaggregate state (b).

FIG. 14(A) shows the fluorescence lifetime decay profile of P1 in THF solution, confirming that the fluorescence intensity decreases over time after excitation at 379 nm wavelength. FIG. 14(B) shows the fluorescence lifetime decay profile of P1 in the H₂O/THF (f_{w}-99 vol %) nanoaggregate state. In the presence of various concentrations of PA, the fluorescence intensity was observed to decrease over time. The inset in the figure shows that the fluorescence lifetimes (τₒ/τ) remain constant or independent of the added PA concentration. Therefore, the fluorescence lifetime of the P1 polymer remains constant even with the addition of PA, confirming consistent quenching behavior.

Hereinafter, the present invention will be described in more detail by way of examples.

These examples are intended to explain the present invention in more detail, and the scope of the present invention is not limited to these examples.

### <Example 1> Production 1 of 2,5-Polysilole P1 Polymer

This Example was carried out according to Reaction Scheme 1 below. A solution of compound 3 (dibutyl bis(phenylethynyl)silane, 5 g, 14.51 mmol) in 50 ml THF was added dropwise to a LiNaph solution over 10 minutes at room temperature. The mixture was stirred for an additional 40 minutes and then cooled to 0° C. ZnCl₂ (7.9 g, 58 mmol) was added to the mixture, followed by dilution with 30 mL THF, thus obtaining intermediate Compound 5 as a black suspension. The mixture was stirred for 40 minutes at room temperature, and Compound 4 (1,4-diiodo-2,5-bis(octyloxy)benzene, 8.5 g, 14.51 mmol) and Pd(PPh₃)₂Cl₂ (102 mg, 1 mol %) were added successively to the mixture, thus synthesizing Compound 6 as the reaction mixture. Compound 7 was synthesized through a one-pot reaction between Compound 6 and excess ZnCl₂, and then the synthesized Compound 7 and Compound 6 were coupled to produce a 2,5-polysilole P1 polymer [¹H NMR spectroscopy (300 MHz, CDCl₃): δ 7.56-6.23 (multiplet, 14H, aromatic protons), 4.0-3.42 (multiplet, 8H, methylene groups) and 1.80-0.86 (multiplet, 78H, alkyl and silane groups). Weight-average molecular weight (Mw)=9,862, number-average molecular weight (Mn)=6,237, and polydispersity index (PDI)=1.581.

### <Example 2> Production 2 of 2,5-Polysilole P2 Polymer

According to Reaction Scheme 1 below, intermediate Compound 5 obtained from Compound 3 was reacted with compound 4 (1,4-diiodo-2,5-bis(octyloxy)benzene) to synthesize Compound 6. Compound 7 was synthesized through a one-pot reaction between Compound 6 and excess ZnCl₂, and then the synthesized Compound 7 and Compound 4 were coupled to produce a 2,5-polysilol P2 polymer [¹H NMR spectroscopy (300 MHz, CDCl₃): δ 7.56-6.23 (multiplet, 16H, aromatic protons), 4.0-3.42 (multiplet, 12H, methylene groups), and 1.80-0.86 (multiplet, 108H, alkyl and silane groups). Weight-average molecular weight (Mw)=4,970, number-average molecular weight (Mn)=3,764, and polydispersity index (PDI)=1.32].

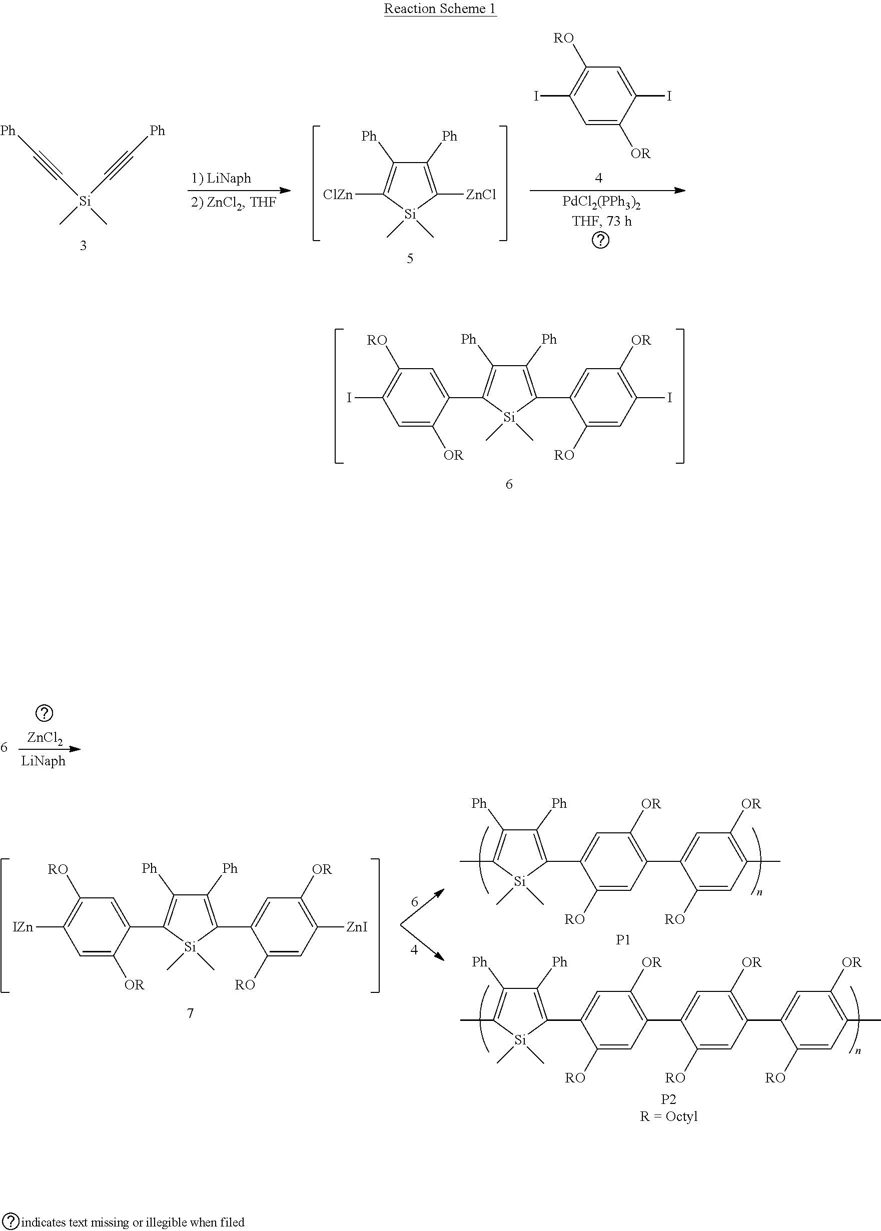

### <Comparative Example 1> Production of 1,1-Polysilole Polymer

A 1,1-polysilole polymer was produced according to Reaction Scheme 2 below.

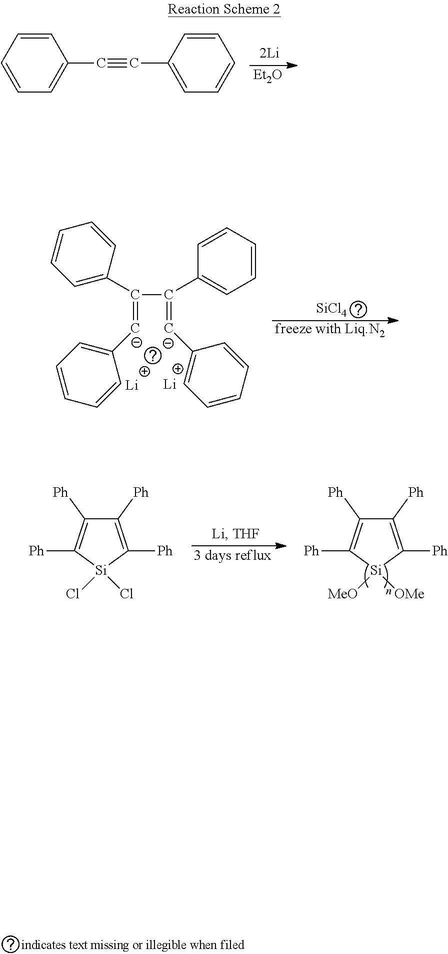

### <Experimental Example 1> Spectroscopic Evaluation

To evaluate the spectroscopic properties of the 2,5-polysilol P1 and P2 polymers produced in Examples 1 and 2 and the 1,1-polysilol compound of Comparative Example 1, UV-VIS absorption spectra and fluorescence properties were evaluated in various states, including solution, film, and aggregate.

TABLE 1

| | λ_{ab} [nm] | λₑₘ [nm] | λₑₘ [nm] | λₑₘ [nm] | Φ_{F} (%) | Φ_{F} (%) | Φ_{F} (%) |
|---|---|---|---|---|---|---|---|
| | Solution | Solution | Film | Aggregate | Solution | Solid | Aggregate |
| Example 1 | 338 | 410 | 503 | 504 | 35.1 | 44 | 39.5 |
| Example 2 | 332 | 405 | 435 | 407 | 33.9 | 15.8 | 19.2 |
| Comparative | 368 | 513 | 513 | 513 | 2.3 | 31.9 | 30.7 |
| Example 1 | | | | | | | |

Example 1: 2,5-polysilole P1 polymer
Example 2: 2,5-polysilole P2 polymer
Comparative Example 1: 1,1-polysilole polymer

Referring to the results in Table 1 above, the 2,5-polysilole P1 polymer of Example 1 exhibited blue emission at 410 nm in the THF solution, while P2 emitted at 405 nm. The maximum absorption wavelength (λ_{ab}) of the P1 and P2 polymers remained consistent in the THF solution due to the same conformation of the polymer backbones, and the maximum emission wavelength (λₑₘ) of the P1 and P2 polymers shifted to a longer wavelength (red-shift) depending on the state of solution→film→aggregate. However, the slight blue shift observed for P2 was attributed to the presence of an additional phenyl linker, which resulted in a slightly twisted structure in the excited state, reducing the long conjugation interaction in the polymer backbone.

On the other hand, the 1,1-polysilole of Comparative Example 1 has a maximum absorption wavelength of 368 nm in solution, and maintains the same emission wavelength of 513 nm in the solution, film and aggregate states.

In particular, the emission peak of P1 polymer showed a red shift of 93 nm in the solid state compared to the THF solution and exhibited a higher absolute quantum yield (QY) value of 44% in the solid state, surpassing that of 1,1-polysilole (31.9%).

FIG. 3 shows the UV-Vis spectra of the 2,5-polysilole P1 polymer of the present invention in THF/H₂O mixtures (0%, 60%, and 90% water fractions), and FIG. 4 shows the UV-Vis spectra of the 2,5-polysilole P2 polymer of the present invention in THF/H₂O mixtures (0%, 60%, 80%, and 90% water fractions).

From the results in FIGS. 3 and 4, it was confirmed that both P1 and P2 polymers did not show significant changes in the maximum absorption wavelength, but as the water fraction increased, the absorption intensity increased, confirming that polymer aggregation was formed in the presence of water.

### <Experimental Example 2> Evaluation of AIEE and ICT Properties

The emission spectra of the 2,5-polysilole P1 and P2 polymers of the present invention were measured in THF and THF/H₂O mixtures to investigate the AIEE and ICT properties thereof.

FIG. 5 shows the PL spectra of the 2,5-polysilole P1 polymer in THF/H₂O with different water fractions, and the insets in FIG. 5 show fluorescence photographs of the polymer taken under 365 nm UV lamp according to THF/H₂O fraction.

As a result, it was confirmed that, in THF solution (f_{w}—0 vol %), P1 emitted at 410 nm and P2 emitted at 405 nm. As the water fraction (f_{w}) increased, the polymers underwent aggregation, resulting in changes in their emission behavior.

FIG. 6 shows the change in relative PL intensity (I/I₀) depending on the water fractions shown in FIG. 5. As can be seen therein, the emission of P1 showed a red shift from 410 nm (f_{w}—0 vol %) to 504 nm (f_{w}—99 vol %) with an increase in solvent polarity. For P1, the emission intensity remained relatively constant up to f_{w}—30%. However, at f_{w}—50%, the emission intensity decreased significantly due to the combined effect of solvent polarity and strong ICT in P1. At this time, P1 did not form colloids up to f_{w}—50%.

Furthermore, as the water fraction (f_{w}) increases, the solvent polarity increases, and the ICT effect becomes more prominent in P1. This result is clearly observed in the solvatochromism, where the emission color of the polymer changes upon addition of water to the solution. Specifically, the increase in f_{w} decreases the emission intensity of P1 up to fw—50 vol %, and at fw≥50 vol %, P1 aggregates, blocking the ICT channel and inducing the AIEE effect.

Consequently, the emission intensity of P1 increased, and the polymer mainly exhibited AIEE behavior. Notably, the PL intensity of the P1 aggregates at f_{w}—99% was 7 times higher than that of the P1 aggregates at f_{w}—60%. These results show that P1 exhibits dual active properties of AIEE and ICT depending on the solvent polarity and aggregation state, and both phenomena contribute to the emission behavior of the polymer.

Similarly, PL spectra of the P2 polymer according to the mole fraction showed quenching behavior due to aggregation (aggregation-caused quenching, ACQ) up to fw—60 vol % due to strong π-π interactions. However, at f_{w}—80 vol % or higher, P2 exhibits AIEE behavior due to the presence of (octyloxy)benzene groups, which prevent π-π stacking interactions in the aggregated state. The restriction of intramolecular rotation (RIR) in the aggregated state blocks non-radiative decay and enhances emission intensity [not shown].

Both the 2,5-polysiole P1 and P2 polymers show ICT behavior due to the presence of electron-donating and electron-accepting units in their molecular structures.

In pure THF solution (f_{w}—0 vol %), the absolute quantum yield (Φ_{F}) values of P1 and P2 are 35.1% and 33.9%, respectively. In contrast, at fw—60 vol %, the ΦF value of P1 decreases to 16%, and in the case of P2, it decreases by 14.7%. However, as the water fraction (f_{w}) gradually increases, the Φ_{F} values of both polymers gradually increase, reaching maximum values of 39.5% for P1 and 19.4% for P2.

P1 shows a remarkable AIE effect with a Φ_{F} value 2.4 times higher than that at f_{w}—60 vol %, while P2 shows a 1.3 times higher Φ_{F} value when it reaches f_{w}—80 vol %.

### <Experimental Example 3> Morphological Evaluation

To demonstrate the aggregation formation and the morphology of the 2,5-polysilole polymers of Examples 1 and 2 in THF/H₂O mixtures, the dynamic light scattering (DLS) measurements and scanning electron microscope (SEM) morphologies were investigated.

FIGS. 7(A)-7(D) show dynamic light scattering (DLS) measurements and scanning electron microscope (SEM) morphologies of the 2,5-polysilole P1 and P2 polymers of the present invention.

According to the DLS results, the average diameters of the particles were about 113 nm for P1 in THF/H₂O (f_{w}—99 vol %) (FIG. 7(A)) and 194 nm for P2 in THF/H₂O (f_{w}—80 vol %) (H₂O fraction: 80%) (FIG. 7(B)), respectively. As the water fraction (f_{w}) increases, the particle size of the aggregates decreases.

In addition, SEM images of P1 (FIG. 7(C)) and P2 (FIG. 7(D)) aggregates in THF/H₂O mixtures confirm the formation of well-dispersed spherical nanoparticles in various aqueous solutions.

### <Experimental Example 4> Evaluation of Solvatochromic Effect

The 2,5-polysilole P1 and P2 polymers of Examples 1 and 2 were evaluated for their solvatochromism behaviors, as well as their emission spectra and absolute fluorescence quantum yields (QYs) in different solvents with varying polarities.

The PL spectra in different solvents indicate that, in hexane, P1 emits at 400 nm and P2 emits at 398 nm. As the solvent polarity increases to moderately polar solvents such as dichloromethane (DCM), the emission peaks red shift to 425 nm for P1 and 413 nm for P2.

Furthermore, when the solvent polarity becomes even higher such as N,N-dimethylformamide (DMF), the emission peaks of P1 and P2 continue to shift to longer wavelengths, reaching 424 and 418 nm, respectively. This solvatochromic shift in emission wavelength is a characteristic of fluorophores with D-A moieties, where the polarity of the solvent affects the stability of the excited state. That is, in highly polar solvents, the excited state of ICT-based polymers is more stable, resulting in a bathochromic shift (red shift) in the emission wavelength. This shift is accompanied by a decrease in quantum efficiency and emission intensity,

FIG. 8 shows fluorescence images of the 2,5-polysilole P1 polymer of the present invention under 365 nm UV light and sunlight in various solvent conditions, visually demonstrating the emission behavior in different solvent environments.

### <Experimental Example 5> XRD Analysis

For the 2,5-polysilole P1 and P2 polymer powders of Examples 1 and 2, powder X-ray diffraction (PXRD) spectra were measured.

FIG. 9 shows the powder X-ray diffraction (PXRD) spectra of the 2,5-polysilole P1 and P2 polymers of the present invention. The PXRD spectra show strong and sharp peaks, indicating that both polymers possess semicrystalline structures.

Specifically, the regular repeating units in the polymer structures contribute to the formation of these crystalline regions. Additionally, broad peaks suggest the presence of an amorphous component, and the broadness of the peaks indicates the presence of regions with disordered or less-defined molecular arrangement within the polymers.

### <Experimental Example 6> Evaluation of Thermal Properties

The thermal properties of the 2,5-polysiole P1 and P2 polymers of Examples 1 and 2 were investigated using thermogravimetric analysis (TGA).

FIG. 10 shows the thermal properties of the 2,5-polysilole P1 and P2 polymer powders of the present invention. As can be seen therein, both the P1 and P2 polymers showed high thermal stability, as almost no weight loss was observed up to a temperature of approximately 300° C. At higher temperatures, a gradual weight loss was observed. Specifically, the TGA curves of P1 and P2 show the weight loss of the polymer with temperature, with P1 showing a 5% weight loss at 348° C. and P2 showing a 5% weight loss at 370° C. These results confirm the excellent thermal stability of the polymers. Additional analysis results show that at a temperature of 600° C., P1 decreased by 66% of its initial weight, while P2 decreased by 81% of its initial weight.

### <Experimental Example 7> Evaluation of Explosive Vapor Sensing Performance in Thin-Film Solid State

For the 2,5-polysilol P1 polymer of Example 1, it was evaluated whether TNT vapors could be detected by fluorescence quenching in the solid state.

TNT powder (1 g) was placed in a glass chamber and allowed to saturate for 1 hour at room temperature. The sensor device, containing polymer P1, was then positioned in front of the chamber with the gate open for 10 seconds to allow exposure to TNT vapors, during which the fluorescence intensity was recorded. Subsequently, the sensor device was removed from the chamber for 20 seconds to observe the recovery process as described above.

FIG. 11 shows the real-time fluorescence intensity profile of the 2,5-polysilole P1 polymer of the present invention for TNT vapor, depicting the quenching (fluorescence intensity decrease) and recovery (fluorescence intensity increase) processes repeated 7 times over 200 seconds. The arrows indicate the points when the TNT vapor causing quenching is introduced and removed. Specifically, when exposed to 5.5 ppb of TNT vapor for 10 seconds at 25° C., the fluorescence intensity decreases in the quenching stage.

After removing the TNT analyte, about 85% of the photoluminescence (PL) intensity was recovered within 10 seconds. These results support that the P1 polymer film can detect TNT vapor through fluorescence quenching, as evidenced by the significant decrease in fluorescence intensity when exposed to TNT. At the same time, the P1 polymer film showed fast self-recovery performance within a very short time after removing the analyte.

### <Experimental Example 7> Evaluation of Explosive Sensing Performance in Solution State and Nanoaggregate State

The explosive sensing performance of the 2,5-polysilol P1 and P2 polymers of the Examples was evaluated by investigating the quenching behavior of the polymers for picric acid (PA) and TNT in both THF solution and aggregate states.

In the presence of the explosives (TNT or PA), the polymers showed fluorescence quenching characteristics in all states. The quenching data were analyzed using the Stern-Volmer equation, which relates the fluorescence intensities in the absence (I₀) and presence (I) of the analyte [A] through the Stern-Volmer constant (Kₛᵥ), and the results are shown in Table 2 below. At this time, for the nanoaggregates, THF/H₂O (f_{w}—99 vol %) was used for P1 and THF/H₂O (f_{w}—80 vol %) was used for P2.

$$( {I}_{0} / I ) - 1 = {K}_{sv} [ A ]$$

TABLE 2

| NAEs | PA (Kₛᵥ, M⁻¹) | | TNT (Kₛᵥ, M⁻¹) | |
|---|---|---|---|---|
| State | Solution | Nanoaggregate | Solution | Nanoaggregate |
| Example 1, P1 | 686 | 2,344 | 127 | 800 |
| Example 2, P2 | 355 | 4,581 | 129 | 770 |

The Stern-Volmer plots showed a linear relationship under all conditions of solution and nanoaggregate states of P1 and P2. As confirmed in Table 2 above, the Stern-Volmer constants (Kₛᵥ) of P1 and P2 nanoaggregates for PA and TNT were observed to be significantly higher than those of the solution state.

In addition, the Stern-Volmer constants of P1 nanoaggregates for PA and TNT were determined to be 2,344 and 800 M⁻¹, respectively. The Stern-Volmer constant of P2 nanoaggregates was similar to that of P1 for TNT, while it was twice as high as P1 for PA.

FIGS. 12(A)-12(B) show PL spectra of the 2,5-polysilole P1 polymer nanoaggregates of the present invention depending on the explosive (a) TNT concentration or (b) PA, showing quenching behavior as the PL spectrum intensity decreases with increasing concentration of TNT or PA. In addition, the P2 nanoaggregate of Example 2 exhibited PL quenching as the concentration of TNT or PA increased.

FIGS. 13(A)-13(B) shows the PL spectra of the 2,5-polysilole P1 solution state according to the concentration of explosives (a) TNT or (b) PA, showing the same result of quenching behavior with increasing concentrations and decreasing PL spectrum intensity.

On the other hand, the Stern-Volmer constants (Kₛᵥ) of the 1,1-polysilole of Comparative Example 1 in the presence of the TNT analyte in the solution and nanoaggregate states showed larger Kₛᵥ values compared to the P1 and P2 nanoaggregates.

These results suggest that the 1,1-polysilole molecule has a flexible molecular structure that can easily interact with explosives, resulting in higher PL quenching. On the other hand, the lower Kₛᵥ values of P1 and P2 can be explained by the reduced intercalation of explosive analytes in both solution and nanoaggregate states.

Therefore, the 2,5-polysilole P1 and P2 polymers with smaller Stern-Volmer constants than 1,1-polysilole can be explained by their relatively non-flexible structure, and the significantly weak binding affinity between polysiloles and explosives (TNT or PA) supports the reversible results of quenching or very fast recovery.

### <Experimental Example 9> Evaluation of Fluorescence Lifetime

To evaluate the fluorescence lifetime of the 2,5-polysilol P1 polymer of the Example, the fluorescence decay of P1 in the solution state and nanoaggregate state was observed for various concentrations of picric acid (PA).

FIG. 14(A) shows the fluorescence lifetime decay profile of P1 in THF solution. The profile likely shows the decay of fluorescence intensity over time after excitation at a wavelength of 379 nm. FIG. 14(B) shows the fluorescence lifetime decay profile of P1 nanoaggregates in a mixture of H₂O and THF (f_{w}—99 vol %). In the presence of various concentrations of PA, the fluorescence intensity was observed to decrease over time. The inset shows the fluorescence lifetime (τₒ/τ) according to concentration, which remains constant or independent of the added PA concentration. Therefore, the fluorescence lifetime of P1 remains constant even with the addition of PA, confirming consistent quenching behavior. This suggests that the quenching process related to fluorescence decay is not dependent on the collision rate of the analyte (PA) and P1 but rather involves a non-dynamic interaction between P1 and PA.

Although the present invention has been described in detail only with respect to the described embodiments, it is apparent to those skilled in the art that various changes and modifications are possible without departing from the technical idea of the present invention. It is to be understood that these changes and modifications fall within the scope of the appended claims.

What is claimed is:

1. A high fluorescence conjugated silol polymer based on 2,5-polysilol, characterized in that it is a silol polymer having a benzene subunit comprising a sterically hindered functional group at the 2,5 positions of the silol backbone, wherein said sterically hindered functional group forms a cavity in the structure of the silol polymer and is rigid.

2. The highly fluorescent conjugated silole polymer of claim 1, which is represented by Formula 1 below: 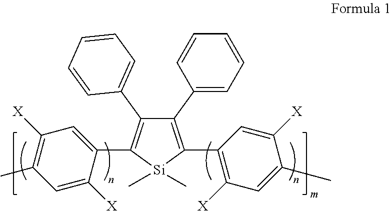
wherein X is a C₂-C₁₂ alkyl group or a C₂-C₁₂ alkoxy group, m is 2 to 20, and n is 1 to 5.

3. The highly fluorescent conjugated silole polymer of claim 1, characterized in that the benzene subunits containing the sterically hindered functional groups (X) is octyloxybenzene subunit.

4. The highly fluorescent conjugated silole polymer according to claim 1, characterized in that the highly fluorescent conjugated silole polymer is a rigid 2,5-polysilole polymer having a semi-crystalline structure.

5. The highly fluorescent conjugated silole polymer of claim 1, which is thermally stable up to a temperature of 300° C.

6. An explosive vapor sensor using the highly fluorescent conjugated silole polymer based on 2,5-polysilole of claim 1.

7. The explosive vapor sensor of claim 6, which exhibits a fluorescence quenching process upon exposure to explosive vapors and a photoluminescence (PL) recovery process upon removal of explosive vapors, which are repeated in real time.

8. The explosive vapor sensor of claim 6, which is useful for detecting nitroaromatic explosives (NAEs).

9. An explosive vapor sensor using the highly fluorescent conjugated silole polymer based on 2,5-polysilole of claim 2.

10. An explosive vapor sensor using the highly fluorescent conjugated silole polymer based on 2,5-polysilole of claim 3.

11. An explosive vapor sensor using the highly fluorescent conjugated silole polymer based on 2,5-polysilole of claim 4.

12. An explosive vapor sensor using the highly fluorescent conjugated silole polymer based on 2,5-polysilole of claim 5.
